(19) United States
(12) Patent Application Publication
(10) Pub. No.: US 2026/0240867 A1
(43) Pub. Date: Aug. 20, 2026

(54) MULTINUCLEAR HETEROCYCLIC METAL CARBENE COMPLEX FOR WOUND HEALING

(71) Applicant: KING ABDULAZIZ UNIVERSITY, Jeddah (SA)

(72) Inventors: Mohammad ASAD, Jeddah (SA); Ayesha RIAZ, Faisalabad (PK); Muhammad Adnan IQBAL, Faisalabad (PK); Haq Nawaz BHATTI, Faisalabad (PK); Muhammad Nadeem ARSHAD, Jeddah (SA); Abdullah M. ASIRI, Jeddah (SA); Khalid A. ALZAHRANI, Jeddah (SA); Naved AZUM, Jeddah (SA)

(73) Assignee: KING ABDULAZIZ UNIVERSITY, Jeddah (SA)

(21) Appl. No.: 19/057,181

(22) Filed: Feb. 19, 2025

(51) Int. Cl. A61K 31/555; A61P 17/02; C07F 1/10

(52) U.S. Cl. CPC A61K 31/555; A61P 17/02; C07F 1/10

(57) ABSTRACT

A complex includes at least two N-heterocyclic carbene (NHC) ligands with the formula I, and two to six silver atoms. R is selected from a group consisting of a propyl functional group, a butyl functional group, and a benzyl functional group. The silver atoms are bonded to the at least two NHC ligands.

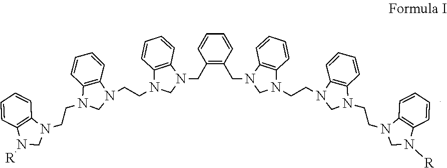

### STATEMENT OF ACKNOWLEDGEMENT

Support provided by the Institutional Fund Projects, Ministry of Education and King Abdulaziz University, DSR, Jeddah, Saudi Arabia, through grant number IFPP-016-130-2022 is gratefully acknowledged.

### BACKGROUND

### Technical Field

The present disclosure is directed to a carbene complex, and more particularly, towards a multinuclear heterocyclic metal carbene complex for wound healing.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

There are currently several methods and techniques for the treatment of cancer, among which may include: radiation therapy, chemotherapy, immunotherapy, and surgery. The shared characteristic of these techniques, as well as other known methods, is that they are extracellular techniques and cancer cells are targeted by applying forces and/or substances outside the cell.

Cancer-related wound healing agents are treatments or compounds developed to address difficulties of wound healing in cancer patients. These challenges often stem from cancer therapies (such as surgery, radiation, and chemotherapy) that hinder the body's natural healing abilities. Moreover, certain cancers can directly impact tissues involved in healing, causing chronic wounds and/or delayed recovery. The aim of these healing agents is to support tissue repair, decrease inflammation, manage infections, and reduce the risk of recurrence and/or complications linked to cancer treatment. Complexes comprising wound healing agents often combine multiple therapeutic agents, including growth factors (e.g., platelet-derived growth factor (PDGF), epidermal growth factor (EGF)), antioxidants (e.g., vitamin C, curcumin), antimicrobial agents (e.g., honey, silver sulfadiazine), and collagen and/or hydrogel-based materials, and work by stimulating cell proliferation, reducing oxidative stress, controlling infection, and supporting tissue regeneration. The complexes aim to accelerate wound healing, prevent complications, and improve recovery, especially in cancer patients with compromised immune systems or chronic, treatment-induced wounds.

Metal carbene complexes have a wide range of applications, including homogeneous catalysis, medicine, dye-sensitized solar cells, and hydrogen (H₂) and gas molecule storage systems. In the field of drug development, metal carbene complexes, particularly those involving platinum, ruthenium, and/or gold, are being investigated for their potential in anticancer, antimicrobial, and antifungal treatments.

Carbene complexes, particularly metal carbene complexes, may be used as anti-tumor, anti-angiogenic, and/or wound-healing agents. The carbene complexes may interfere with DNA replication, generate reactive oxygen species (ROS), and/or induce apoptosis in cancer cells, making them effective anti-tumor agents. Carbene complexes may inhibit angiogenesis by targeting pathways like vascular endothelial growth factor (VEGF) and limit blood supply to tumors. In wound healing, carbene complexes may promote tissue regeneration, reduce inflammation, and have antimicrobial effects, aiding recovery, particularly in cancer patients with impaired immune function.

New anti-cancer medications have focused on gold N-heterocyclic carbene (Au—NHC) complexes; however, the Au—NHC complexes have demonstrated undesired toxicity in vivo. Further, Au—NHC complexes are costly in nature. Cisplatin has widespread use in chemotherapy treatments; however, it has several side effects, including nephrotoxicity, hair loss, neurotoxicity, and diarrhea. Copper carbene complexes also have disadvantages as wound-healing agents due to potential toxicity from excessive copper release, instability in biological environments, and the risk of inducing inflammation and/or oxidative stress. Limited bioavailability and the need for additional antimicrobial agents may reduce its effectiveness in complex wound healing. Current carbene complexes suffer from one or more drawbacks hindering their adoption into widely applicable medical treatments.

Accordingly, an object of the present disclosure is to provide a metal carbene complex that may circumvent drawbacks such as, high toxicity, low stability, low bioavailability, complex synthesis procedure, and/or high cost of the materials known in the art.

### SUMMARY

In an exemplary embodiment, a complex is described. The complex includes at least two N-heterocyclic carbene (NHC) ligands with the formula I:

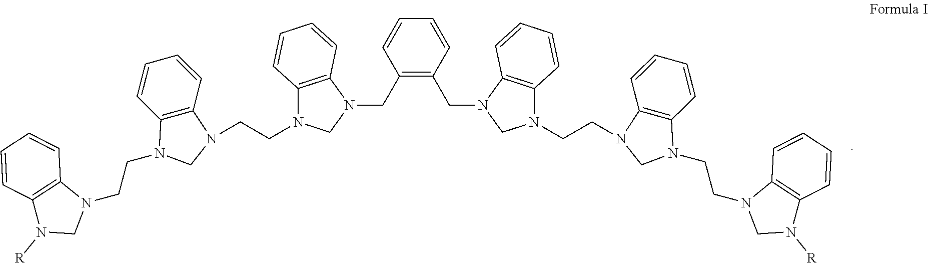

R is selected from a group consisting of a propyl functional group, a butyl functional group, and a benzyl functional group. The complex includes two to six silver atoms and each of the silver atoms are bonded to the at least two NHC ligands.

In some embodiments, the NHC ligand of formula I is made by a process, including refluxing a 1-X-1H-benzimidazole with a 1,2-dibromoethane to form a 3-(2-bromoethyl)-1-X-1H-benzoimidazolium, where X is selected from a group consisting of propyl, butyl, and benzyl. The method further includes refluxing a 1,3-bis((1H-imidazol-1-yl)methyl)benzene with the 3-(2-bromoethyl)-1-X-1H-benzoimidazolium in an organic solution to form a tetrakis-benzamidazolium precursor. The method includes refluxing the tetrakis-benzamidazolium precursor with 3-(2-bromoethyl)-1-X-1H-benzoimidazolium in 1,4-dioxane to form a salt of formula 1.

In some embodiments, X is propyl and the tetrakis-benzamidazolium precursor has a percent yield of 45 to 50% based on an initial weight of reactants.

In some embodiments, X is butyl and the tetrakis-benzamidazolium precursor has a percent yield of 42 to 48% based on an initial weight of reactants.

In some embodiments, X is benzyl and the tetrakis-benzamidazolium precursor has a percent yield of 45 to 50% based on an initial weight of reactants.

In some embodiments, the complex is made by a process including mixing the NHC ligand of formula I with a silver salt in a polar solvent in the absence of light for 45 to 50 hours (h) to form a mixture, followed by filtering the mixture, collecting a filtrate, and mixing a hexafluorophosphate salt with the filtrate in a polar solvent to form the complex.

In some embodiments, a method of healing wounds includes administering the complex to an open wound and closing the open wound to form a healed wound. A surface area of the healed wound is smaller compared to an initial surface area of the open wound.

In some embodiments, administering the complex to the open wound occurs at least 2 times a day.

In some embodiments, administering the complex to the open wound occurs for at least 1 to 15 days.

In some embodiments, after administering the complex to the open wound and the closing the open wound, the wound heals with a percent wound contraction of 70 to 85% based on the initial surface area of the open wound.

In some embodiments, a method of anti-angiogenesis is described. The method includes administering the complex to a subject and reducing growth of blood vessels in the subject.

In some embodiments, the method further includes dissolving blood vessels during the reducing.

In some embodiments, the complex further includes at least two NHC ligands with the formula II:

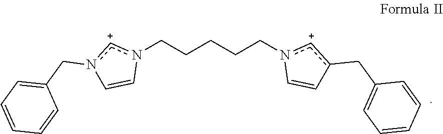

In some embodiments, the complex further includes at least two NHC ligands with the formula III:

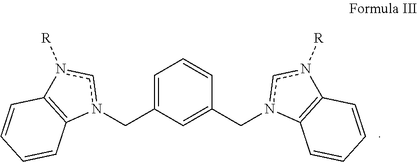

R is selected from a group consisting of a propyl functional group, a butyl functional group, a hexyl functional group, a benzyl functional group, and an ethyl phenyl functional group.

In some embodiments, the complex further includes at least two NHC ligands with the formula IV:

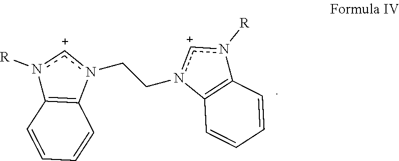

R is a diphenyl methane functional group.

In some embodiments, the complex has a percent yield of 75 to 85% based on an initial weight of reactants.

In some embodiments, the complex has a percent yield of 65 to 90% based on an initial weight of reactants.

In some embodiments, the complex has a percent yield of 80 to 85% based on an initial weight of reactants.

In some embodiments, the functional group is the hexyl functional group, and the complex has a unit cell volume of 1650 to 1700 cubic angstroms (Å³).

In some embodiments, a pharmaceutical composition is described. The composition includes the complex, a filler, a stabilizer, a surfactant, a binder, a glidant, and a lubricant.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is an exemplary flow chart of a method of making a N-heterocyclic carbene (NHC) ligand, according to certain embodiments.

FIG. 1B is an exemplary flow chart of a method of making a complex, according to certain embodiments.

FIG. 1C is an exemplary flow chart of a method of healing of wounds with the complex, according to certain embodiments.

FIG. 1D is an exemplary flow chart of a method of anti-angiogenesis, according to certain embodiments.

FIG. 2 is a chorioallantoic membrane (CAM) assay of selected complexes before incubation showing clear vessel growth in chick embryo formation, according to certain embodiments.

FIG. 3 is a CAM assay of selected complexes after 24 hours incubation showing the dissolution of vessels due to antiangiogenic activity, according to certain embodiments.

FIG. 4 depicts wounds on a first day after administering the complex depicting a 15×15-millimeter (mm) wound, according to certain embodiments.

FIG. 5 depicts contracted wounds on a 14^{th} day after administering the complex with wound contraction, according to certain embodiments.

FIG. 6 is a graph depicting mean±standard deviation (sd) of wound contraction (%) at a second day, according to certain embodiments.

FIG. 7 is a graph depicting mean±sd of wound contraction (%) at a 7^{th} day, according to certain embodiments.

FIG. 8 is a graph depicting mean±sd of wound contraction (%) at a 14^{th} day, according to certain embodiments.

FIG. 9 is a crystal structure of complex 8, according to certain embodiments.

FIG. 10 is a crystal structure of complex 9, according to certain embodiments.

FIG. 11 is a crystal structure of complex 10, according to certain embodiments.

FIG. 12 is a crystal structure of complex 11, according to certain embodiments.

### DETAILED DESCRIPTION

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all, embodiments of the disclosure are shown.

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “compound” refers to a chemical entity, regardless of its phase (solid, liquid, or gaseous) and/or its state: crude mixture, purified, or isolated.

As used herein, the term “reactant” refers to a substance that takes part in and undergoes change during a reaction.

As used herein, the term “ultrasonication” or “sonication” refers to the process in which sound waves are used to agitate particles in a solution.

As used herein, the term “room temperature” refers to a temperature range of 25+3 degrees Celsius (° C.) in the present disclosure.

As used herein, “filtration” refers to a mechanical and/or physical operation that can be employed for the separation of constituents of homogeneous and/or heterogeneous solutions. Types of filtration can be categorized by the estimated sizes of constituents to be separated and can involve particle filtration (>10 μm), microfiltration (0.1-10 μm), ultrafiltration (0.01-0.1 μm), nanofiltration (NF) (0.001-0.01 μm), and/or reverse osmosis (RO) (<0.001 μm). Other types of filtration may include, but are not limited to, gravity filtration, membrane filtration, ion exchange, cake filtration, centrifugal filtration, evaporation, cold filtration, pressure filtration, multilayer filtration, a combination thereof, and the like.

As used herein, the term “functional group” indicates specific groups of atoms within a molecular structure that may be accountable for characteristic chemical reactions and/or chemical properties of that molecular structure. Suitable examples of functional groups include hydrocarbons, groups containing halogens (fluoro-, chloro-, bromo-, iodo-), groups containing oxygen (alcohol, acid, ketone, aldehydes), groups containing nitrogen (nitrile, amines, amides), groups containing silicon (silanes), groups containing phosphorus, groups containing sulfur, combinations thereof, and all identifiable groups by a skilled person. As used herein, the term “attach” or “attachment” refers to linking or uniting by a bond, link, force (including an intramolecular force and an intermolecular force), and/or tie in order to keep two or more parts together. Attachment encompasses both direct and/or indirect attachment such that, for example, a first compound may be directly bound to a second compound and/or material. In embodiments where one or more intermediate compounds and/or molecules may be disposed between the first compound and the second compound or material.

As used herein, the term “aromatic compounds” or “aromatic rings,” refers to hydrocarbon rings that, by the theory of Hückel, have a cyclic, delocalized (4n+2) pi-electron (n-electron) system. Non-limiting examples of aromatic compounds include benzene, benzene derivatives, compounds having at least one benzene ring in their chemical structure, cyclohexane, cyclohexane derivatives, compounds having at least one cyclohexane ring in their chemical structure, cyclopentene, cyclopentene derivatives, compounds having at least one cyclopentene ring in their chemical structure, cyclopentane, cyclopentane derivatives, compounds having at least one cyclopentane ring in their chemical structure, a combination thereof, and the like.

As used herein, the term “N-heterocyclic carbene (NHC) ligands” refers to organic molecules that contain a carbene group bonded to nitrogen in a nitrogen-containing (N-containing) heterocycle. These ligands are known for their strong donor properties and stability. The structure of an NHC ligand typically consists of a heterocyclic ring (often a 5-membered ring) with nitrogen atoms and a central carbon atom in a carbene group having a formal negative charge, which can act as a strong electron donor to transition metals. As used herein, the term “carbene” refers to a molecule containing a neutral carbon atom with a valence of two and two unshared valence electrons. The general formula of a carbene is R—:C—R′ or R═C, where “R” represents substituents or hydrogen atoms and “:” represents two valence electrons.

As used herein, the term “propyl functional group” refers to a three-carbon alkyl group with the chemical formula C₃H₇. A propyl functional group is derived from propane (C₃H₈) by removing one hydrogen atom. There are two main types of propyl groups: n-Propyl (normal propyl): a straight-chain structure, where the three-carbon atoms are connected in a linear fashion; and isopropyl: a branched structure, where two methyl groups are attached to the second carbon of the chain.

As used herein, the term “butyl functional group” refers to a four-carbon alkyl group derived from butane. Butane has several isomers, such as n-butyl, isobutyl, sec-butyl, and tert-butyl, each differing in their chain or branching structure.

As used herein, the term “benzyl functional group” refers to a phenyl group attached to a methylene group. The benzyl functional group is derived from toluene and is achieved by removing one hydrogen atom from the methyl group. The benzyl group is found in organic chemistry, especially in aromatic compounds, and is known for its ability to stabilize cations and interact with other chemical groups in various reactions. It is distinct from the phenyl group because it includes an additional methylene group.

As used herein, the term “hexyl functional group” refers to a six-carbon alkyl group derived from hexane and achieved by removing one hydrogen atom. It is commonly found as a substituent in organic compounds, contributing hydrophobic characteristics to molecules. The hexyl functional group is a straight-chain alkyl group with no branching in its structure.

As used herein, the term “diphenylmethane functional group” consists of a methane molecule where two phenyl groups are attached to a central carbon atom. It has a general structure of C₆H₅—CH₂—C₆H₅, where the two phenyl rings (benzene rings) are bonded to the same carbon atom through a methylene group.

As used herein, the term “pharmaceutical composition” refers to a mixture of compounds and/or pharmaceutically acceptable salts, esters, and/or prodrugs thereof, with other chemical components, such as physiologically acceptable carriers and excipients.

As used herein, the term “refluxing” refers to a technique where a reaction mixture is heated to its boiling point, and a vapor is condensed back into a liquid phase and returned to a reaction vessel. This process allows a reaction to proceed at an elevated temperature without losing any solvents or reactants, maintaining a constant volume and promoting the reaction to occur under controlled conditions for an extended period.

As used herein, the term “treating” and “treatment” refers to a prophylactic treatment. Treatment methods may include administering a therapeutically effective amount of an active agent or ingredient to a subject. The administering step may consist of a single administration or may include a series of administrations. The length of the treatment period may depend on a variety of factors, such as severity of a condition, age of a patient/subject, concentration of active agents, activity of compositions used in the treatment, and/or a combination thereof. It may also be appreciated that an effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be used. For example, compositions may be administered to the subject/patient in an amount and for a duration sufficient to treat the subject/patient. In some embodiments, the treating or treatment may not be prophylactic treatment.

As used herein, the term “effective amount” refers to an amount sufficient for a compound to accomplish a stated purpose relative to the absence of the compound (e.g., achieve an effect for which it is administered, treat a disease, reduce enzyme activity, increase enzyme activity, reduce a signaling pathway, and/or reduce one or more symptoms of a disease or condition). An example of an “effective amount” is an amount sufficient to contribute to a treatment, prevention, and/or reduction of a symptom or symptoms of a disease, which may also be referred to as a “therapeutically effective amount.”

A weight percent of a component, unless specifically stated to the contrary, is based on a total weight of the formulation or composition in which the component is included. For example, if a particular element or component in a composition or article is said to have 5 weight percentage (wt. %), it is understood that this percentage is in relation to a total compositional percentage of 100%.

The present disclosure is intended to include all hydration states of a given compound and/or formula, unless otherwise noted or when heating a material.

The present disclosure is intended to include all isotopes of atoms occurring in the present compounds and complexes. Isotopes include those atoms having the same atomic number but different mass numbers. By way of general example, and without limitation, isotopes of hydrogen include deuterium and tritium, and isotopes of carbon include ¹³C and ¹⁴C. Isotopes of oxygen include ¹⁶O, ¹⁷O, and ¹⁸O. Isotopically-labeled compounds of the disclosure may generally be prepared by conventional techniques known to those skilled in the art and/or by processes analogous to those described herein, using an appropriate isotopically-labeled reagent in place of the non-labeled reagent otherwise employed.

Aspects of the present disclosure are directed toward multinuclear metal NHC complexes with applications in wound healing. It involves the synthesis of sterically tuned tetra- and hexa-nuclear Ag(I)-NHC complexes, which demonstrate anti-angiogenic, antioxidant, and wound healing properties. The present disclosure describes synthetic methods for these complexes and their therapeutic potential in promoting wound healing. The present disclosure introduces compounds, methods, and compositions aimed at improving wound healing outcomes.

Aspects of the present disclosure are directed toward a complex including at least two NHC ligands with the formula I:

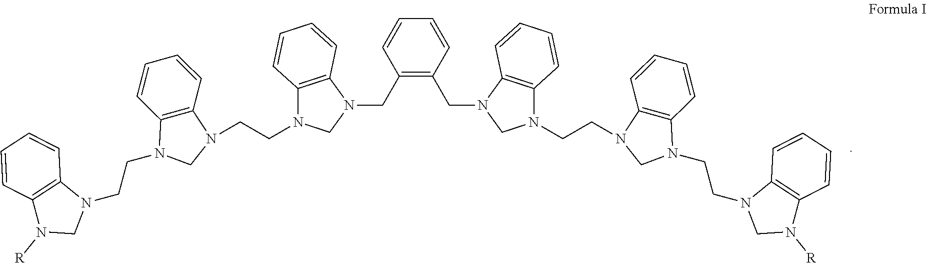

where R is selected from a group including a propyl functional group, a butyl functional group, and a benzyl functional group. The complex includes two to six silver atoms, preferably two silver atoms, preferably three silver atoms, preferably four silver atoms, preferably five silver atoms, and preferably six atoms. Each of the silver atoms are bonded to the at least two NHC ligands. In some embodiments, the silver atoms are bound to a carbon atom in one or more carbenes in the at least two NHC ligands.

In some embodiments, other elements such as gold (Au), platinum (Pt), palladium (Pd), ruthenium (Ru), copper (Cu), nickel (Ni), and/or zinc (Zn) may also be used in combination instead of silver.

In some embodiments, the complex further includes at least two NHC ligands with the formula II:

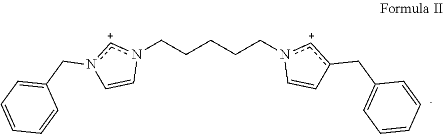

In some embodiments, the complex further includes at least two NHC ligands with the formula III:

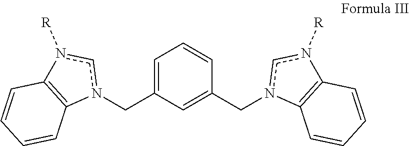

where R is selected from a group including a propyl functional group, a butyl functional group, a hexyl functional group, a benzyl functional group, and an ethyl phenyl functional group.

In some embodiments, the complex further includes at least two NHC ligands with the formula IV:

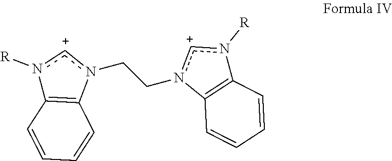

where R is a diphenyl methane functional group. In some embodiments, R may be the same throughout the complex or may be used in combination with one or more different R from a group. In some embodiments, the complex including at least two NHC ligands can be modified by changing alkyl groups and/or functional groups.

In some embodiments, overall ligand charges of the at least two NHC ligands having the formula I, II, III, and IV may be neutral, negative, or positive and may depend on a counterion and/or salt of the NHC ligands. In some embodiments, charges of carbons in a carbene group of an NHC ligand may vary depending on protonation of the carbons and/or a salt form of the NHC ligand.

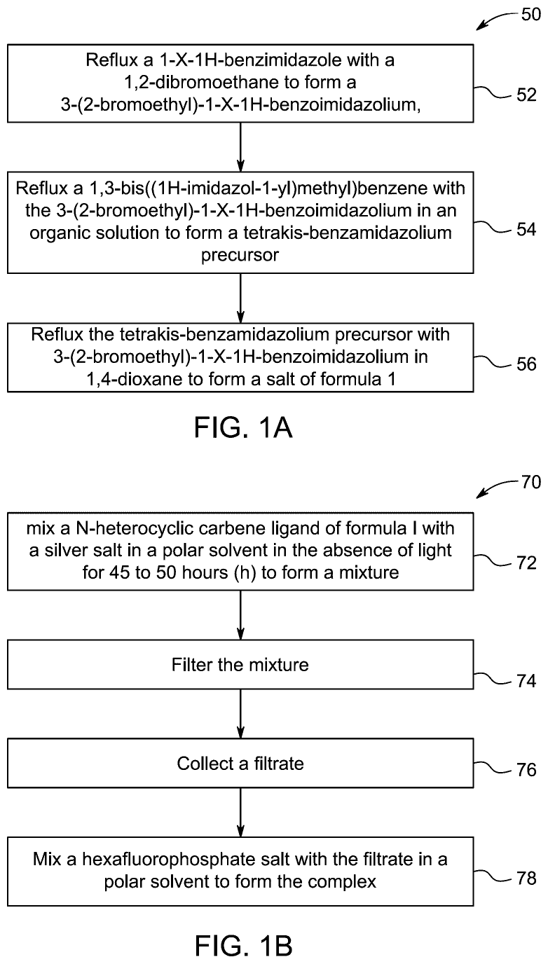

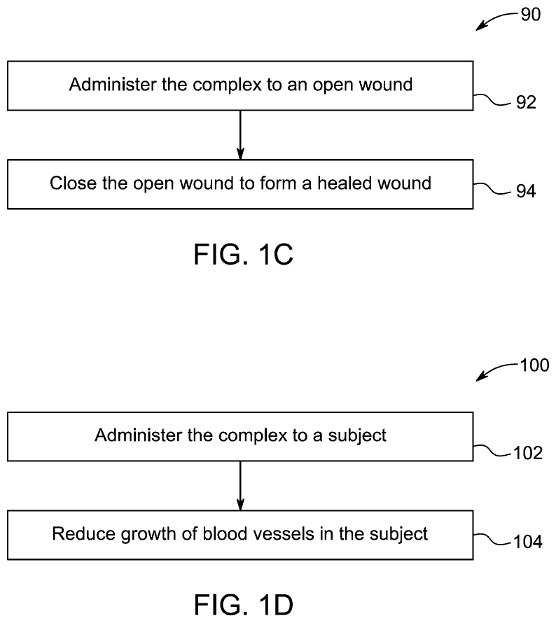

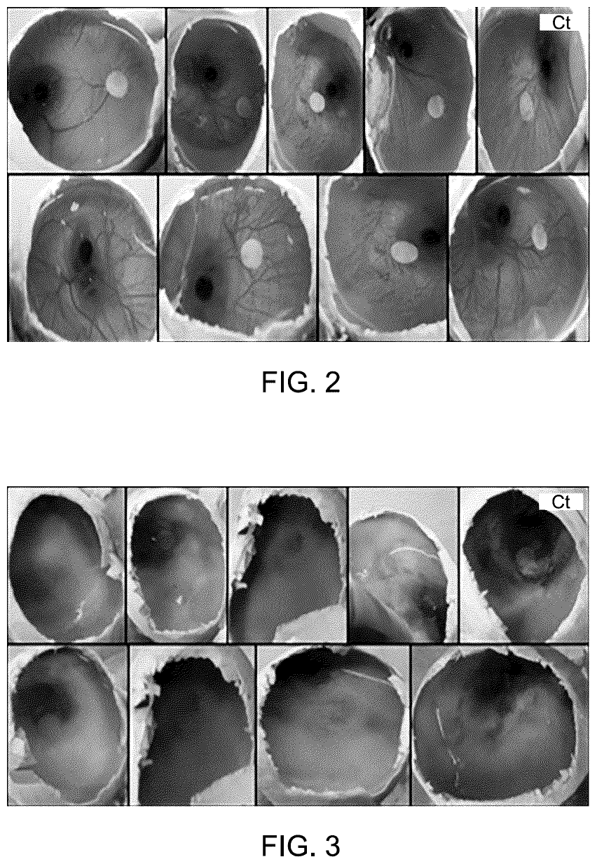

FIG. 1A illustrates a schematic flow chart of a method 50 of making the NHC ligand of formula I. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes refluxing a 1-X-1H-benzimidazole with a 1,2-dibromoethane to form a 3-(2-bromoethyl)-1-X-1H-benzoimidazolium. X is selected from a group including propyl, butyl, and benzyl. In some embodiments, X is propyl and the tetrakis-benzamidazolium precursor has a percent yield of 45-50%, preferably 46-49%, and more preferably 47-48%, based on an initial weight of reactants. In some embodiments, X is butyl and the tetrakis-benzamidazolium precursor has a percent yield of 42-48%, preferably 43-47%, preferably 44-46%, and more preferably about 45%, based on an initial weight of reactants. X is benzyl and the tetrakis-benzamidazolium precursor has a percent yield of 45 to 50%, preferably 46-49%, and more preferably 47-48%, based on an initial weight of reactants.

At step 54, the method 50 includes refluxing a 1,3-bis((1H-imidazol-1-yl)methyl)benzene with the 3-(2-bromoethyl)-1-X-1H-benzoimidazolium in an organic solution to form a tetrakis-benzamidazolium precursor. In some embodiments, the organic solution may include, but is not limited to, tetrahydrofuran, ethyl acetate, dimethylformamide, acetonitrile, acetone, dichloromethane, toluene, dimethyl sulfoxide, nitromethane, propylene carbonate, ethanol, formic acid, n-butanol, methanol, ethanol, a combination thereof, and the like. In a preferred embodiment, the organic solvent is methanol.

At step 56, the method 50 includes refluxing the tetrakis-benzamidazolium precursor with 3-(2-bromoethyl)-1-X-1H-benzoimidazolium in 1,4-dioxane to form a salt of formula 1. Refluxing may accelerate the reaction by providing consistent heating, ensure good solubility of reactants, prevent solvent evaporation, promote efficient formation of the desired salt, and help in purifying the product by removing volatile impurities. This process may improve reaction yield and efficiency. In some embodiments, pyridinium, pyrazolium, triazolium, thiazolium, tetrazolium, quinazolium precursors may also be used in place of or in combination with imidazoline and benzoimidazoline.

FIG. 1B illustrates a flow chart of a method 70 for making the complex. The order in which the method 70 is described is not intended to be construed as a limitation, and any number of the described method steps can be combined in any order to implement the method 70. Additionally, individual steps may be removed or skipped from the method 70 without departing from the spirit and scope of the present disclosure.

At step 72, the method 70 includes mixing the NHC ligand of formula I with a silver salt in a polar solvent in the absence of light for 45-50 hours (h), preferably 46-49 h, and more preferably 47-48 h to form a mixture. A “polar solvent” is a solvent that has a dipole moment, allowing it to dissolve ionic and/or polar compounds. Suitable examples of polar organic solvents include, but are not limited to, methanol, ethanol, isopropanol, 1-butanol and acetone dimethylformamide, diethylformamide, acetonitrile, dimethyl sulfoxide (DMSO), dimethylacetamide (DMA), acetaldehyde, ethyl acetate, methyl ethyl ketone (MEK), tetrahydrofuran (THF), propylene carbonate, N-methyl-2-pyrrolidone (NMP), 1,4-dioxane, formamide, glycol ethers, mixtures thereof, and the like. In a preferred embodiment, the polar solvent is methanol.

In some embodiments, the mixing may be done by stirring, swirling, sonicating, vortexing, a combination thereof, and any mixing methods known may be employed. The silver salt may be silver nitrate, silver chloride, silver sulfate, silver acetate, silver bromide, silver iodide, silver oxide, silver phosphate, a combination thereof, and the like. In a preferred embodiment, the silver salt is silver oxide. Silver oxide acts as a base. In some embodiments, the NHC ligand can react with silver oxide to form mono-, bis-, tri-, tetra-, penta-, or hexa-NHC-Ag(I) complexes.

At step 74, the method 70 includes filtering the mixture. Filtration may be done using a filter paper, by centrifugation, internal and external filtration, gravity filtration, vacuum filtration, pressure filtration, membrane filtration, decantation, gas flotation, capacitance-based separation, microfiltration, a combination thereof, and the like. Alternate techniques, which may be used in other embodiments, for separation include natural and forced sedimentation, magnetic separation, vacuum distillation, chemical conversion, chromatography, and the like.

At step 76, the method 70 includes collecting a filtrate. In some embodiments, the mixture is filtered using a Buchner funnel under vacuum, collecting the filtrate in a flask. In other embodiments, gravity filtration with filter paper in a standard funnel is used to separate a solid residue from the filtrate. In further embodiments, the mixture may be passed through a Celite bed or a sintered glass filter, collecting the filtrate in a separate container. Additionally, in some embodiments, the solid particles may be separated from a liquid using pre-wet filter paper in a funnel, or the filtrate is collected in a separation funnel after settling solids.

At step 78, the method 70 includes mixing a hexafluorophosphate salt with the filtrate in a polar solvent to form the complex. Exemplary hexafluorophosphate salts include, but are not limited to, lithium hexafluorophosphate (LiPF₆), sodium hexafluorophosphate (NaPF₆), potassium hexafluorophosphate (KPF₆), tetraethylammonium hexafluorophosphate (TEA-PF₆), N-benzylpyridinium hexafluorophosphate (BzPy-PF₆), N-methylpyridinium hexafluorophosphate (MePy-PF₆), a combination thereof, and the like. In a preferred embodiment, the hexafluorophosphate salt is KPF₆. Exemplary polar solvents include, but are not limited to, methanol, ethanol, isopropanol, 1-butanol and acetone dimethylformamide, diethylformamide, acetonitrile, DMSO, DMA acetaldehyde, ethyl acetate, MEK, THF, propylene carbonate, NMP, 1,4-dioxane, formamide, glycol ethers, mixtures thereof, and the like. In a preferred embodiment, the polar solvent is methanol.

FIG. 1C illustrates a flow chart of a method 90 for healing wounds. The order in which the method 90 is described is not intended to be construed as a limitation, and any number of the described method steps can be combined in any order to implement the method 90. Additionally, individual steps may be removed or skipped from the method 90 without departing from the spirit and scope of the present disclosure.

At step 92, the process 90 includes administering the complex to an open wound. In some embodiments, the administering the complex to the open wound occurs at least 2 times a day, preferably 3 times a day, and preferably 4 times a day. In some embodiments, the administering the complex to the open wound occurs for at least 1 to 15 days, preferably 2 to 14 days, preferably 3 to 13 days, preferably 4 to 12 days, preferably 5 to 11 days, preferably 6 to 10 days, preferably 7 to 9 days, and preferably about 8 days. As used herein, the term “administering” means oral administration, administration as a suppository, topical contact, intravenous, parenteral, intraperitoneal, intramuscular, intralesional, intrathecal, intranasal, subcutaneous administration, the implantation of a slow-release device, (e.g., a mini-osmotic pump), a combination thereof, and any administration methods known, to a subject. Administration may be by any route, including parenteral and transmucosal (e.g., buccal, sublingual, palatal, gingival, nasal, vaginal, rectal, and/or transdermal). Parenteral administration may include intravenous, intramuscular, intra-arteriole, intradermal, subcutaneous, intraperitoneal, intraventricular, and intracranial, a combination thereof and the like. Other modes of delivery include, but are not limited to, the use of liposomal formulations, intravenous infusion, transdermal patches, and the like. In some embodiments, the administering does not include administration of any active agent other than the complex. In some embodiments, the administration of the complex is selected from a group including intravenous, interperitoneal, intramuscular, oral administration, combinations thereof, and the like.

A pharmaceutical composition, including the complex of the present disclosure, may be administered orally, systemically, parenterally, by inhalation spray, rectally, and/or topically in dosage unit formulations containing conventional, non-toxic pharmaceutically acceptable carriers, adjuvants, and vehicles as desired. In some embodiments, the method of administration of the complex or an analogue or derivative thereof is oral. In other embodiments, the complex or an analogue or derivative thereof is administered by injection, such as, for example, through a peritumoral injection.

Topical administration may also involve the use of transdermal administration, such as transdermal patches and/or iontophoresis devices. The term parenteral, as used herein, includes intravesical, intradermal, transdermal, subcutaneous, intramuscular, intralesional, intracranial, intrapulmonary, intracardial, intrasternal, sublingual injections, and infusion techniques.

Injectable preparations, for example, sterile injectable aqueous or oleaginous suspensions, may be formulated according to the known art using suitable dispersing or wetting agents and/or suspending agents. The sterile injectable preparation may also be a sterile injectable solution and/or suspension in a nontoxic, parenterally acceptable diluent or solvent, for example, as a solution in 1,3-butanediol. Among acceptable vehicles and solvents that may be employed include water, Ringer's solution, isotonic sodium chloride solution, and the like. In addition, sterile, fixed oils may be conventionally employed as a solvent and/or a suspending medium. For this purpose, any fixed oil can be employed, including synthetic mono- and/or diglycerides. In addition, fatty acids, such as oleic acid, find use in the preparation of injectables. Dimethyl acetamide, surfactants including ionic and non-ionic detergents, and polyethylene glycols may also be used. Mixtures of solvents and/or wetting agents may also be useful. Suppositories for rectal administration of the complex or an analogue or derivative thereof may be prepared by mixing the complex or an analogue or derivative thereof with a suitable non-irritating excipient, such as cocoa butter, synthetic mono- di- and/or triglycerides, fatty acids, polyethylene glycols that are solid at ordinary temperatures but liquid at the rectal temperature and will therefore melt in the rectum and release the drug, combinations thereof, and the like.

Solid dosage forms for oral administration can include capsules, tablets, pills, powders, granules, and the like. In such solid dosage forms, the complexes may be combined with one or more adjuvants appropriate to the indicated route of administration. If administered, a contemplated complex or an analogue or derivative thereof may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone (PVP), polyvinyl alcohol (PVA), combinations thereof, and the like, and then tableted and/or encapsulated for convenient administration. Such capsules or tablets may contain a controlled-release formulation, as can be provided in a dispersion of an active compound in hydroxypropyl methylcellulose. In the case of capsules, tablets, and pills, the dosage forms can also include buffering agents, such as sodium citrate, magnesium and/or calcium carbonate, magnesium and/or calcium bicarbonate, combinations thereof, and the like. Tablets and pills may additionally be prepared with enteric coatings.

For therapeutic purposes, formulations for parenteral administration can be in the form of aqueous or non-aqueous isotonic sterile injection solutions and/or suspensions. These solutions and suspensions can be prepared from sterile powders and/or granules having one or more of the carriers and/or diluents mentioned for use in formulations for oral administration. A complex or an analogue or derivative thereof of the present disclosure can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art and may be used in the current disclosure.

Liquid dosage forms for oral administration can include pharmaceutically acceptable emulsions, solutions, suspensions, syrups, and elixirs containing inert diluents commonly used in the art, such as water. Such compositions can also include adjuvants, such as wetting agents, emulsifying and suspending, agents, and sweetening, flavoring, and perfuming agents.

At step 94, the method 90 includes closing the open wound to form a healed wound. In some embodiments, a surface area of the healed wound is smaller compared to an initial surface area of the open wound. After the administering the complex to the open wound, the closing the wound heals with a percent wound contraction of 70-85%, preferably 71-84%, preferably 72-83%, preferably 73-82%, preferably 74-81%, preferably 75-80%, preferably 76-79%, and preferably 77-78% based on an initial surface area of the open wound.

FIG. 1D illustrates a flow chart of a method 100 of anti-angiogenesis. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes administering the complex to a subject. Anti-angiogenesis refers to the process of inhibiting the formation of new blood vessels (angiogenesis). It is a therapeutic strategy used to prevent the growth of tumors and other diseases that rely on the development of new blood vessels for their growth and spread. By blocking angiogenesis, the supply of oxygen and nutrients to tumors is restricted, which can help slow down or stop their growth. In some embodiments, a patient and/or a subject refers to a living organism. Non-limiting examples include humans, other mammals, (i.e., dogs, cats, bovines, rats, mice, dogs, monkeys, goat, sheep, cows), and other non-mammalian animals. In some embodiments, the patient/subject is a cat or a dog. In other embodiments, the patient/subject is a mammal. In yet other embodiments, the patient/subject is a primate. In a preferred embodiment, the patient/subject is a human.

At step 104, the method 100 includes reducing growth of blood vessels in the subject. The complex can also be used for treating cancer cells by inhibiting or reducing growth of blood vessels in the subject. As used herein, the term “cancer” refers to all types of cancer, neoplasm, and/or malignant tumors found in mammals (e.g., humans), including, but not limited to, leukemias, lymphomas, carcinomas, and sarcomas. Exemplary cancers that may be treated with the method and/or complex provided herein include, but are not limited to, brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with the method provided herein include, but are not limited to, cancer of the thyroid, endocrine system, brain, breast, cervix, colon, head and neck, liver, kidney, lung, ovary, pancreas, rectum, stomach, and uterus. Additional examples include, but are not limited to, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

In some embodiments, the complex further includes at least two NHC ligands with the formula II. In some embodiments, the complex has a percent yield of 75-85%, preferably 76-84%, preferably 77-83%, preferably 78-82%, preferably 79-81%, based on an initial weight of reactants.

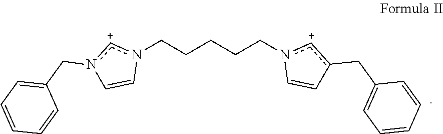

In some embodiments, the complex further includes at least two NHC ligands with the formula III:

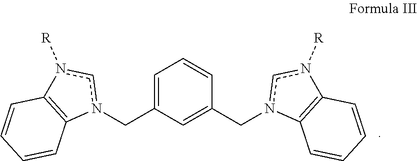

In some embodiments, R is selected from a group including a propyl functional group, a butyl functional group, a hexyl functional group, a benzyl functional group, and an ethyl phenyl functional group. In some embodiments, the complex has a percent yield of 65-90%, preferably 66-89%, preferably 67-88%, preferably 68-87%, preferably 69-86%, preferably 70-85%, preferably 71-84%, preferably 72-83%, preferably 73-82%, preferably 74-81%, preferably 75-80%, preferably 76-79%, and preferably 77-78%, based on an initial weight of reactants. In some embodiments, the functional group is the hexyl functional group, and the complex has a unit cell volume of 1650 to 1700 cubic angstroms (Å³), preferably 1660-1690 Å³, preferably 1670-1680 Å³, and yet more preferably about 1678.96 Å³. In some embodiments, the unit cell may have cubic, tetragonal, orthorhombic, rhombohedral, hexagonal, monoclinic, and triclinic geometries. In a preferred embodiment, the unit cell has monoclinic and triclinic geometries. In some embodiments, the complex has a percent yield of 75 to 85%, preferably 77 to 83%, preferably 78 to 82%, more preferably 79 to 81%, and yet more preferably about 80%.

In some embodiments, the complex further includes at least two NHC ligands with the formula IV. In an embodiment, R is a diphenyl methane functional group. In some embodiments, the complex has a percent yield of 80-85%, preferably 81-84%, and more preferably 82-83%.

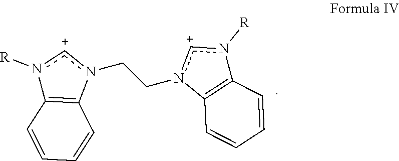

In some embodiments, the complex also acts as an antioxidant compound. In some embodiments, the complex may be used to inhibit the growth of microbes such as bacteria (e.g., Staphylococcus aureus, Bacillus subtilis, Escherichia coli, Pseudomonas aeruginosa) and fungi (e.g., Candida albicans).

In some embodiments, a pharmaceutical composition is described. The pharmaceutical composition includes the complex, a filler, a stabilizer, a surfactant, a binder, a glidant, and a lubricant. A filler is an inert substance added to pharmaceutical formulations to increase volume and ensure proper dosing, particularly when an active ingredient is potent or in small quantities. Examples of fillers may include, but are not limited to, lactose, cellulose, mannitol, a combination thereof, and the like. A stabilizer prevents the degradation of an active ingredient due to factors such as heat, light, and/or moisture, helping to maintain the drug's potency and shelf-life. Common stabilizers may include, but are not limited to, ascorbic acid, EDTA, and the like. A surfactant may reduce surface tension, enhancing the solubility and/or absorption of not readily soluble drugs. Examples of surfactants may include, but are not limited to, polysorbate 80, sodium lauryl sulfate, and the like. A binder holds tablet or capsule ingredients together, ensuring cohesion and structural integrity. Examples of binders include, but are not limited to, PVP, hydroxypropylmethylcellulose (HPMC) polyvinylidene fluoride (PVDF)-based polymers, its co- and terpolymers with hexafluoroethylene, tetrafluoroethylene, chlorotrifluoroethylene, chlorotrifluoroethylene, polyvinyl fluoride, styrene-butadiene rubber, polytetrafluoroethylene (PTFE), ethylene chlorotrifluoroethylene (ECTFE), ethylene tetrafluoroethylene (ETFE), fluorinated-ethylene-propylene (FEP), perfluoro-alkoxy (PFA), polychlorotrifluoroethylene (PCTFE), sulfonated tetrafluoroethylene (Nafion®), polybutadiene, cyanoethyl cellulose, carboxymethyl cellulose, polyacrylonitrile, ethylene propylene diene terpolymers (EPDM), polyimides, ethylene-vinyl acetate copolymers, combinations thereof, and the like. A glidant improves the flow properties of powders, reducing friction during manufacturing. Examples of glidants include, but are not limited to, talc, corn starch, colloidal silicon dioxide, and the like. A lubricant reduces friction between the tablet and manufacturing equipment, promoting smooth ejection. Examples of lubricants may include, but are not limited to, magnesium stearate, stearic acid, and the like.

In addition to fillers, stabilizers, surfactants, binders, glidants, and lubricants, pharmaceutical compositions may also contain disintegrants (e.g., starch, croscarmellose sodium) to aid tablet breakup, colorants (e.g., titanium dioxide) for appearance, preservatives (e.g., phenol, methylparaben) to prevent microbial growth, sweeteners (e.g., sucrose, aspartame) for taste, flavors (e.g., peppermint oil, fruit flavors) to mask bitterness, antifoaming agents (e.g., simethicone) for foam control, pH adjusters (e.g., citric acid, sodium hydroxide) to maintain stability, humectants (e.g., glycerin) to retain moisture, coatings (e.g., enteric coatings, sugar coatings) for protection and controlled release, solvents (e.g., water, ethanol) for dissolution, and any other additives known in the art. These excipients may improve the formulation's stability, efficacy, and patient compliance.

### EXAMPLES

The following examples describe and demonstrate a complex comprising two N-heterocyclic carbene (NHC) ligands and silver. Examples are provided solely for illustration and are not to be construed as limitations of the present disclosure, as many variations thereof are possible without departing from the spirit and scope of the present disclosure.

### Example 1: Synthesis of formula 5 [(1,3-bis ((1H-benzimidazole-1-yl)methyl)benzene) (P₁)]

Imidazolium salts are precursors of NHC. Imidazolium salts may be transformed into its equivalent NHC by a variety of methods. Imidazolium salts may be converted into NHC by deprotonating them with a base, like potassium hydroxide (KOH), in dimethyl sulfoxide (DMSO).

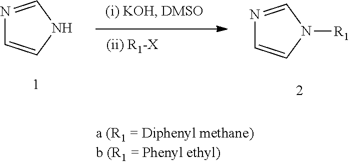

The reaction of benzimidazole (formula 3) with m-xylene dichloride (formula 4) in the presence of KOH in DMSO as a solvent resulted in compound 1,3-bis((1H-benzimidazole-1-yl)methyl)benzene P1 represented by the formula 5 in 88% yield.

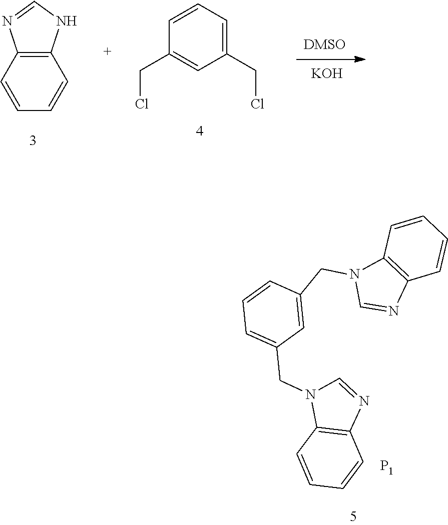

### Example 2: Synthesis of formula 8 [3-(2-bromoethyl)-1-propyl-1H-benzomidazoleium bromide (P₂ₐ), 3-(2-bromoethyl)-1-butyl-1H-benzo[d]imidazolium (P_{2b}), and 3-(2-bromoethyl)-1-benzyl-1H-benzoimidazolium (P_{2c})]

Synthesis of 3-(2-bromoethyl)-1-propyl-1H-benzomidazoleium bromide (P₂ₐ) includes solvent free reaction of N-propyl benzimidazole (formula 7) with 1, and subsequent reflux with 2-dibromoethane to result in compound P₂ₐ (formula 8) in 51% yield. The precursor has been characterized by Fourier-transform infrared spectroscopy (FTIR) and nuclear magnetic resonance (NMR).

A similar reaction using N-butyl imidazole (formula 7) with 1,2-dibromoethane forms 3-(2-bromoethyl)-1-butyl-1H-benzo[d]imidazolium (P_{2b}) (formula 8) in 52% yield. The precursor has been characterized by FTIR and NMR.

Combination of N-benzyl imidazole (formula 7) with 1,2-dibromoethane to form 3-(2-bromoethyl)-1-benzyl-1H-benzoimidazolium (P_{2c}) (formula 8) with a yield of 42%. Both FTIR and NMR have been used to characterize the precursor.

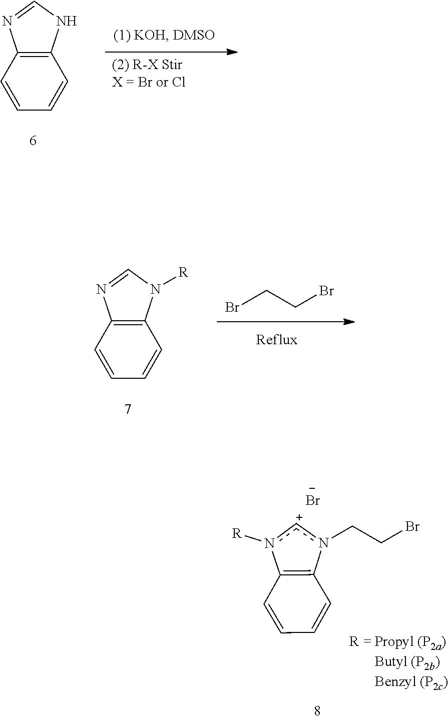

### Example 3: Synthesis of S1, S2 and S3

The combination of 1 equivalent of an N-alkylated imidazole was added to 1,4-dioxane containing alkyl halide (1 equivalent), refluxed, and a product was formed. Product formation was examined by gas chromatography mass spectrometry (GC-MS). The reaction of N-diphenyl methylimidazole and 1-bromooctane in 1,4-dioxane gave the S1 product (formula 11) in 82% yield.

The reaction of N-diphenyl methyl imidazole and 1-bromohexane in 1,4-dioxane gave the S2 product (formula 11) in 81% yield.

The combination of 1 equivalent of N-phenethyl imidazole with 2-bromoethyl benzene in 1,4-dioxane resulted in S3 product (formula 11) in 68% yield.

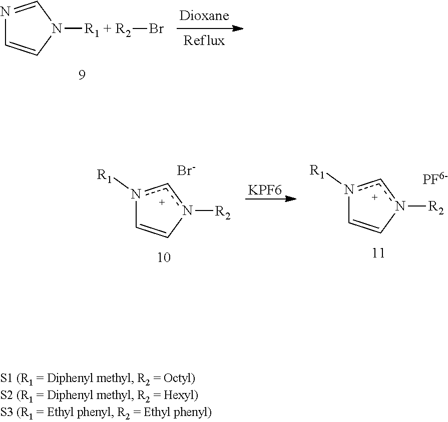

### Example 4: Synthesis of Compound 13

N-alkylated benzimidazole (1 equivalent) was added to 1,4-dioxane containing alkyl halide (1 equivalent) and refluxed to obtain product 13. Product formation was examined by GC-MS.

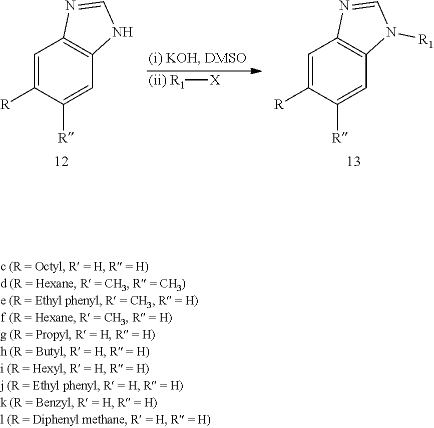

### Example 5: Synthesis of S4 and S5

The combination of compound 13 using N-octyl benzimidazole (c) and 1-bromooctane in 1,4-dioxane gave the product S4 of formula 16 in 92% yield. After 48 h reflux, product was settled down as white powder.

The S5 product (87% yield) was synthesized by combination of compound 13 using N-hexyl 5,6-dimethyl-benzimidazole (d) and 1-bromohexane in 1,4-dioxane.

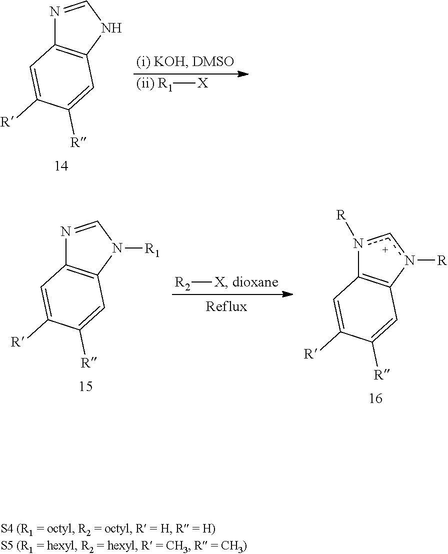

### Example 6: Synthesis of S6 and S7

The reaction of N-alkylated 5-methyl-benzimidazole (e) with 2-bromoethyl benzene in 1,4-dioxane gave the S6 product (formula 19) product in a yield of 75%. The reaction of N-hexyl methyl-substitute benzimidazole and 1-bromohexane in 1,4-dioxane gave the S7 product (formula 19) in a 68% yield.

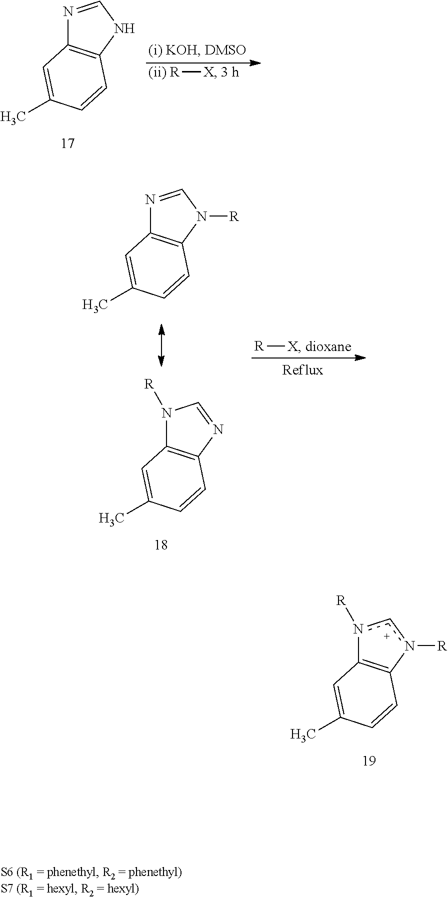

### Example 7: Synthesis of S8

N-benzyl imidazole (formula 20) was added to 1,4-dioxane containing 1,5-dibromopentae (formula 21), then refluxed for 2 days to give the S8 product (formula 22) with a yield of 78%. Product formation was examined by GC-MS.

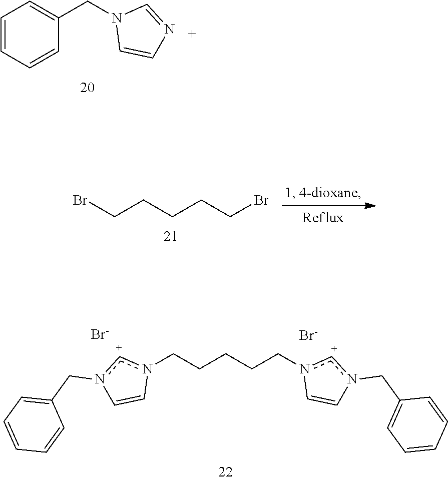

### Example 8: Synthesis of S8, S9, S10, S11, S12, S13 and S14

The combination of N-propyl benzimidazole (g) and 1,3-bis(chloromethyl)benzene in fresh 1,4-dioxane gave the S9 product (formula 24) with a product yield of 82%. The S10 product (in 81.2% yield) was synthesized by the combination of N-butyl benzimidazole (h) and 1,3-bis(chloromethyl)benzene in 1,4-dioxane. The combination of N-hexyl benzimidazole (i) and 1,3-bis(chloromethyl)benzene in 1,4-dioxane gave the product S11 (formula 24) in 76% yield.

Product S12 (formula 24) was formed by using N-benzyl benzimidazole (k) and 1,3-bis(chloromethyl)benzene) in 1,4-dioxane with an 81% yield. The S13 product (formula 24) was synthesized using N-phenethyl benzimidazole (j) and 1,3-bis(chloromethyl)benzene in 1,4-dioxane with a 71% yield. The combination of N-diphenylmethyl benzimidazole (1) and 1,3-bis(chloromethyl)benzene in fresh 1,4-dioxane gave the S14 product (formula 24) in a 78% yield.

In case of S14, a product was not precipitated. A chloride salt was converted to a hexafluorophosphate counterpart by a metathesis reaction using potassium hexafluorophosphate (KPF₆) (2 equivalent) in a methanol/water (MeOH/H₂O) system. The metathesis mixture was kept stirring for 4 hours (h). After that, a precipitated product was filtered, washed (with water several times), and dried. The product was collected in the form of a white powder.

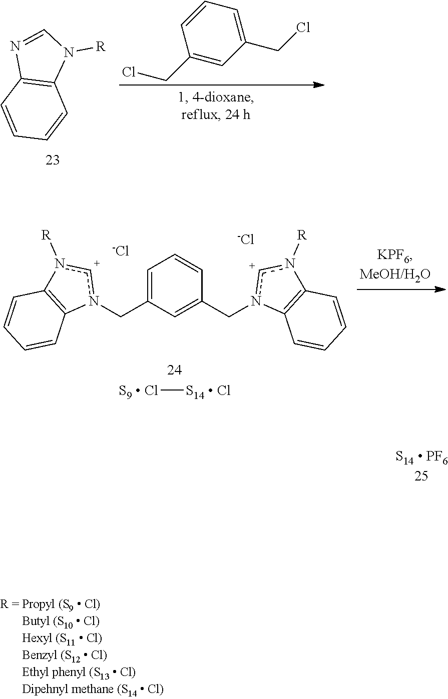

### Example 9: Synthesis of S15 and S16

The S15 product (formula 27) was formed using N-butyl benzimidazole (h) and 1,2-dibromoethane in 1,4-dioxane in a 68% yield. The combination of N-hexyl benzimidazole (i) and 1,2-dibromoethane in 1,4-dioxane gave the S16 product (formula 27) in a 75% yield.

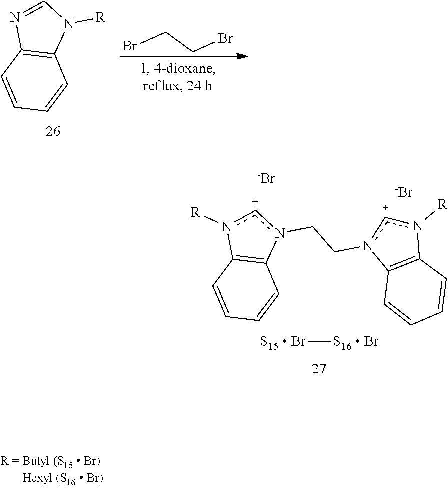

### Example 10: Synthesis of tetrkis-bezimidazolium salts (S17, S18, S19)

1 equivalent of precursor 1 (formula 5) was added to hot 1,4-dioxane containing precursor 2 (a-c) (formula 8) and a small amount of methanol (MeOH). The reaction mixture was refluxed for 24 h, after which a white powder settled down at bottom of a reaction flask. The obtained product was filtered and washed with fresh 1,4-dioxane, dichloromethane, and diethyl ether (Et₂O). Pure salts (S17-S19) were collected as white powder.

Tetrkis-bezimidazolium salts were synthesized by the interaction of benzimidazolium precursors with 1,4 dioxane in methanol (MeOH). The combination of 1 equivalent of formula 5 with 0.5 equivalent of 8 (P₂ₐ, P_{2b}, and P_{2c}) gave the formula 28 in 48%, 45%, and 48% yield, respectively. Synthesis of the salts have been reported [Riaz, A. et al., Synthesis of sandwich type acyclic tetra-nuclear silver(I)-N-heterocyclic carbene complexes for wound healing applications, Z Naturforsch C J Biosci, 2020, 75, 9-10, 369-376, which is incorporated herein by reference in its entirety].

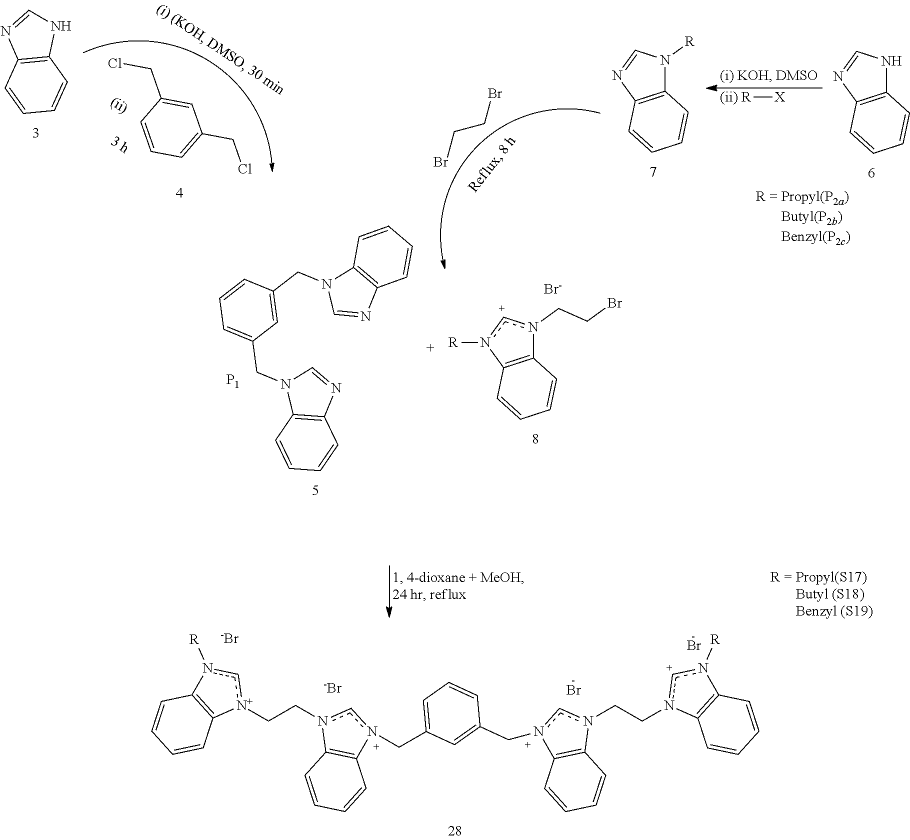

### Example 11: Synthesis of $20, S21, and S22

The combination of benzimidazolium precursor (formula 8) with tetrakis-benzamidazolium precursor (formula 29) at room temperature results in hexa-bezimidazolium salts. Synthesis of propyl substituted hexakis benzimidazolium bromide salt (S20), butyl substituted hexakis benzimidazolium bromide salt (S21) and benzyl substituted hexakis benzimidazolium bromide salt (S22) are achieved.

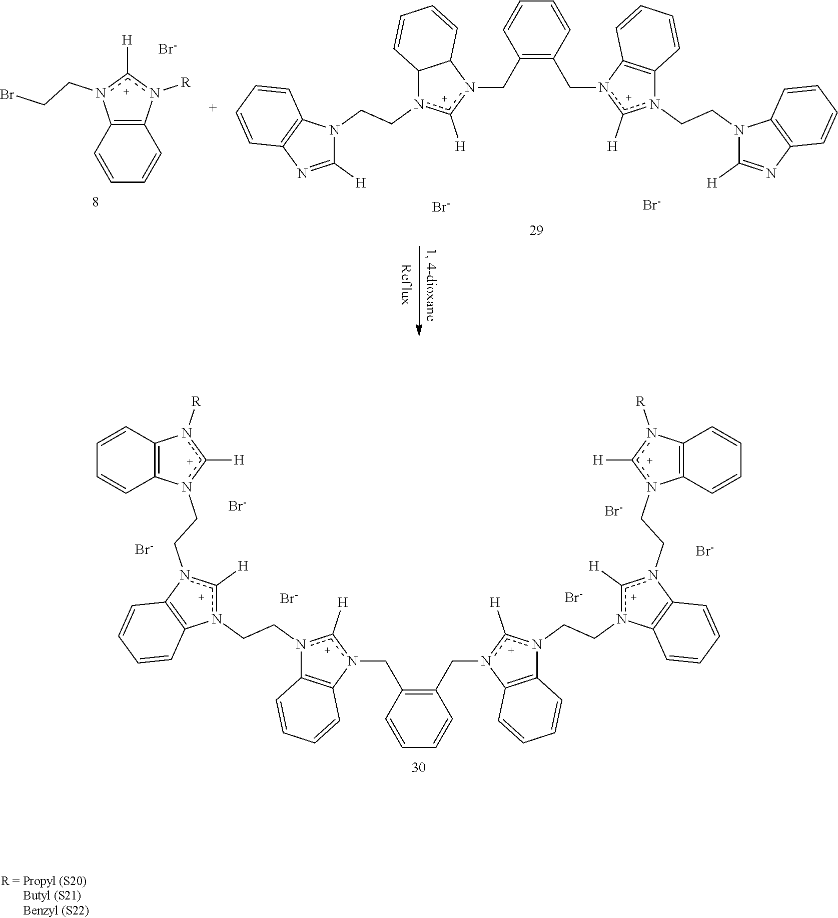

Metalation was done in a round bottom flask covered with aluminum foil. Imidazolium/benzimidazolium salts were reacted with silver(I) oxide (Ag₂O) in methanol (80 milliliters (mL)) by stirring for 2 days at room temperature. The reaction mixtures were filtered through celite twice to get a crystal-clear solution. For metathesis, KPF₆ (2 equivalent) dissolved in water was gradually added to the solution and shaken again for 3-4 h until precipitation was achieved. The precipitate was filtered, washed, and dried. White silvery powder was collected.

### Example 12: Synthesis of Carbene Silver Complexes (C1, C2 and C3)

The combination of compound 11 with Ag₂O resulted in the formation of silver carbene complexes represented by the formula 31 (C1, C2 and C3) in yields of 75%, 72% and 78%, respectively.

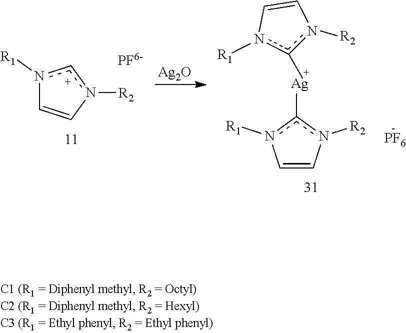

### Example 13: Synthesis of C4 and C5

Ag₂O and S4 (formula 16) were reacted to produce C4 (formula 31) with a yield of 81%. By combining S4 with Ag₂O, a similar synthetic route was employed to produce the C5 (formula 32) with an 86% yield.

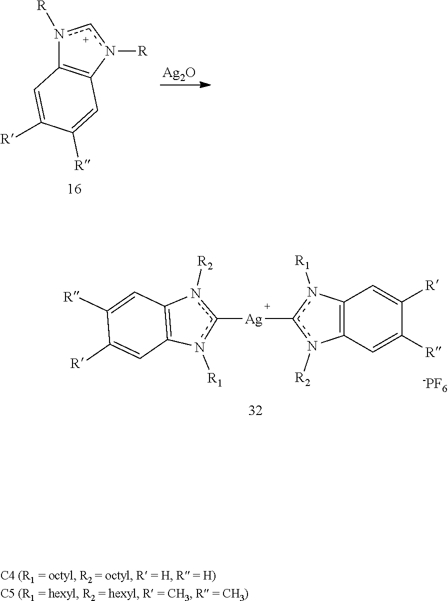

### Example 14: Synthesis of C6 and C7

A similar reaction was carried out for C6 and C7 products (formula 33) by a combination of S6 and S7, respectively, (formula 19) with Ag₂O with 72% and 73% yields, respectively.

### Example 15: Synthesis of Silver(I)-NHC Complex (C8)

Silver(I)-NHC complex, C8 (formula 34), was formed by combination of S8 (formula 22) with Ag₂O in an 80% yield.

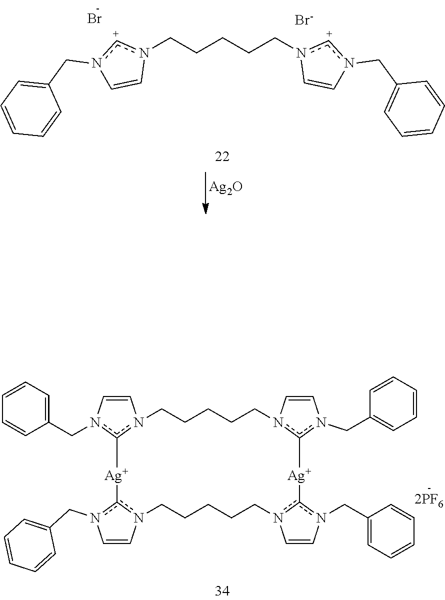

### Example 16: Synthesis of Silver(I)-NHC Complex (C9, C10, C11, C12, and C13)

For synthesis of silver(I)-NHC complexes C9, C10, C11, C12, and C13 (formula 35), a combination of formula 25 with Ag₂O results in 86%, 83%, 80%, and 68% yields, respectively.

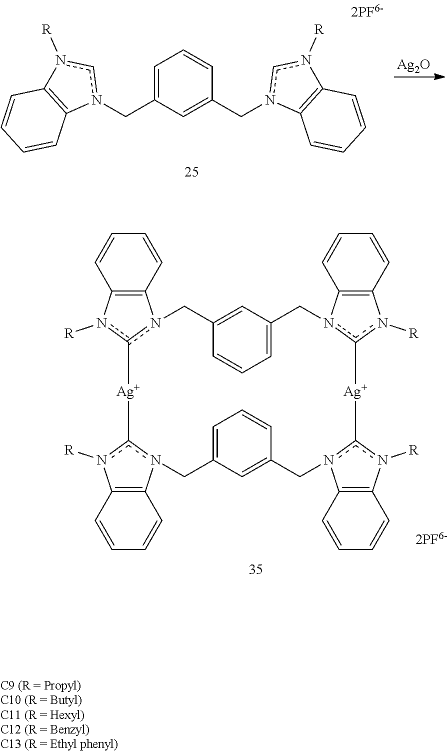

### Example 17: Synthesis of C14

The combination of formula 25 with Ag₂O gave the C14 product (formula 36) in an 82% yield.

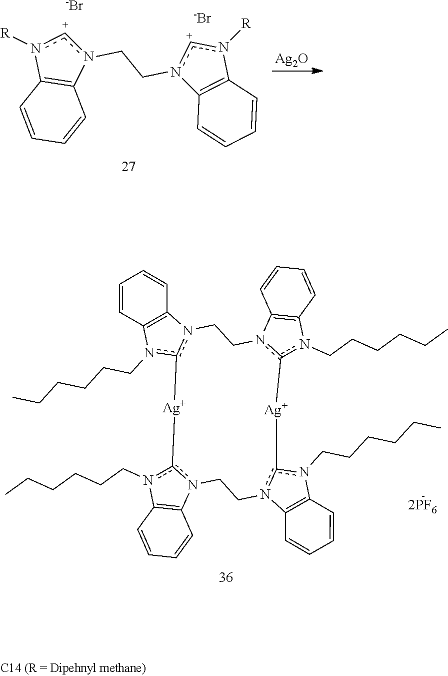

A general synthesis of silver(I)-hexaNHC complexes was carried out by adding multinuclear benzimidazolium bromide salts in a suspension of silver oxide in MeOH and stirred for 48 h in the absence of light. The black suspension was filtered through celite. The halide counter ion was exchanged with PF₆⁻ ions by a metathesis reaction under continuous stirring. Immediately, precipitation occurred. The preiciptates were left to stir for 4 h, filtered, washed with distilled water and dried.

### Example 18: Synthesis of C15, C16, and C17

C15, C16, and C17 products (formula 37) were formed by combination of S20, S21, and S22 products (formula 29), respectively, with silver oxide in MeOH.

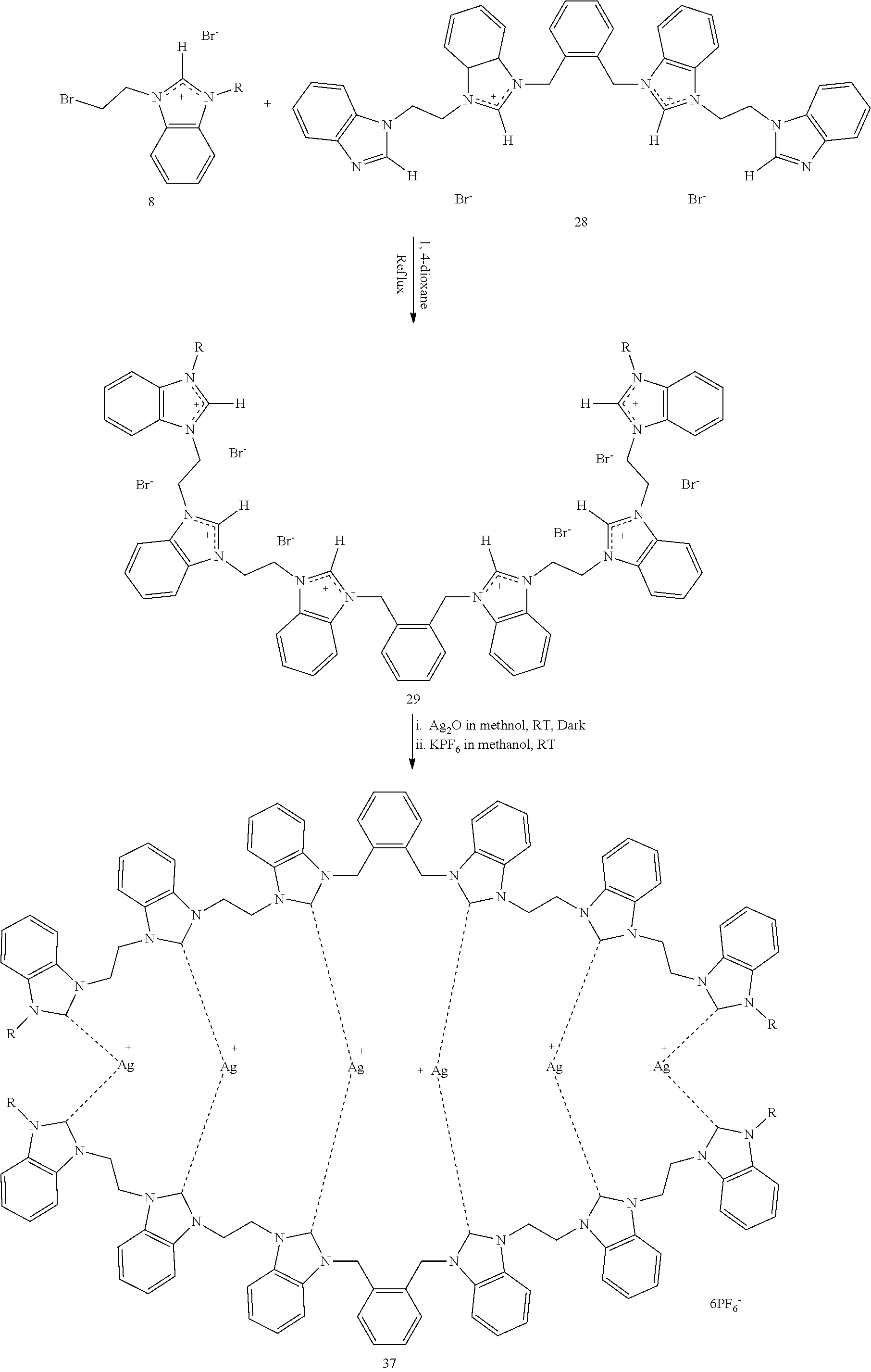

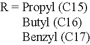

### Example 19: Antioxidant Activity

Percent inhibition of scavenging activity of salts and complexes was determined by a 2,2-diphenyl-1-picrylhydrazyl (DPPH) assay using ascorbic acid as positive control and DMSO as a negative control. Equation 1 was used to determine the percentage inhibition of free radical scavenging activity.

$$Inhibition ( \% ) = [ 1 - ( {A}_{s} / {A}_{c} ) ] \times 100 \qquad ( 1 )$$

Aₛ is the absorbance of sample well and Ac is the absorbance of control well.

Compounds showed antioxidant activity at concentrations of 200 micromolar (μM) in the range of 5-25% for Ag(I) complexes and 20-33% for NHC salts. Samples with low concentrations did not show free radical scavenging activity with some compounds showing zero activity at low concentrations. Results indicate that the synthesized salts and complexes may not be potent antioxidant agents. The findings were in accordance with reported antioxidant activities of Ag complexes.

### Example 20: Anti-Angiogenic Effect

Angiogenic activity of Ag complexes was evaluated by an in ovo chick chorioallantoic membrane (CAM) assay. The assay was performed to determine anti-angiogenic properties of the complexes. Solutions of complexes and ligands in DMSO were used as treatments and DMSO alone was used as a negative control. After 24 h incubation time, the CAM assay results were observed and photographed. The synthesized compounds successfully inhibited the blood vessels growth, which means they may be effective in inhibition of solid tumor growth in cancer.

CAM assay results of complexes before and after incubation were compared. There were clear growing vessels observed in all CAM assays before incubation. FIG. 2 is a chorioallantoic membrane (CAM) assay of selected complexes before incubation showing clear vessel growth in chick embryo formation. After incubating the CAMs for 24 h, no dissolution of vessels were observed in the control, as shown in FIG. 3. The dissolution of growing vessels in treated CAMs demonstrate the anti-angiogenic effect of the silver complexes.

### Example 21: Wound Contraction Effect

In addition to anti-angiogenic and antioxidant activities, the present disclosure observes a wound contraction effect. Seven silver(I) complexes were selected from each series of complexes and their wound healing abilities were determined. A 14 day study was done by making a 10% paste of complexes in a neutral base cream from a commercial source. Healthy domestic rabbits weighing 1.5-2 kilograms were used for the wound contraction effect. Wounds (three per animal) of 15×15 millimeters (mm) were created on a dorsal surface of rabbit skin with a scalpel blade. Treatments were applied in triplicate on individual animal two times a day. For a control no treatment was applied, while commercially available “Contractobex gel” was used for a standard treatment.

Every day, each of the wounds of the animals were checked, and percentage wound contraction was observed. The effectiveness of all complexes was shown for wound healing; however, the wound healing ability of complexes were good for complexes with 2 silver ions and better for 4 silver ions. Benzimidazolium-based complexes were found to be more efficient than imidazolium-based complexes. This may be because of good biological activity of benzimidazole.

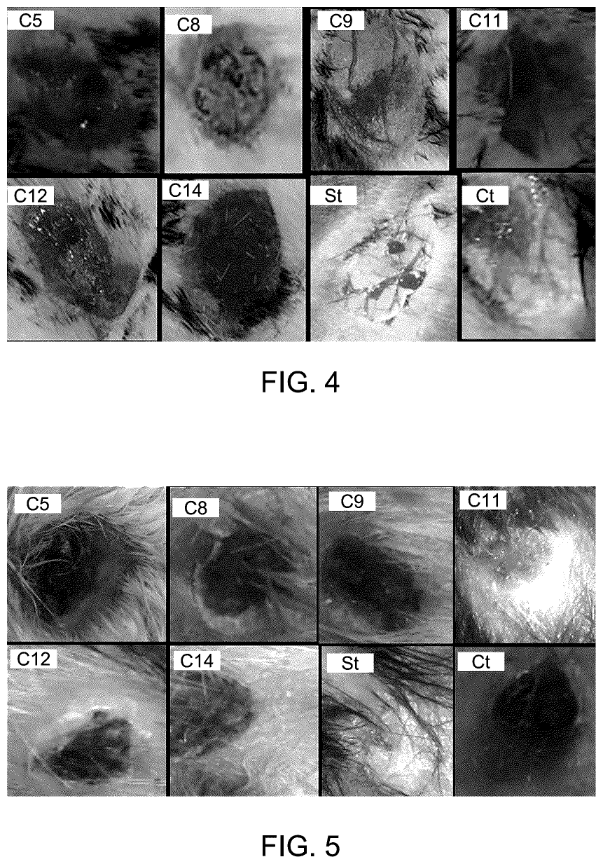

FIG. 4 depicts wounds on a first day with a 15×15 millimeter (mm) wound. Bis- and hexakis-NHC-Ag complexes showed activity comparable to commercially available wound contracting gel. Wounds of the negative control animal healed at a much slower rate compared to the bis- and hexakis-NHC-Ag complexes. Wounds with no treatment showed a wound similar in size to the initial wound size, with contraction of the wounds taking place at a slow rate. Wounds treated with C5 (formula 31) and C8 (formula 33) were not contracted much, but C9, C11, C12, and C14 (formula 34, 35, and 36) showed much better wound contraction, with the highest percentage of wound contraction observed for C14 (formula 36), as shown in FIG. 4. Contracted wounds on the 14th day showed the control sample with the largest wound size area compared to the others, as shown in FIG. 5.

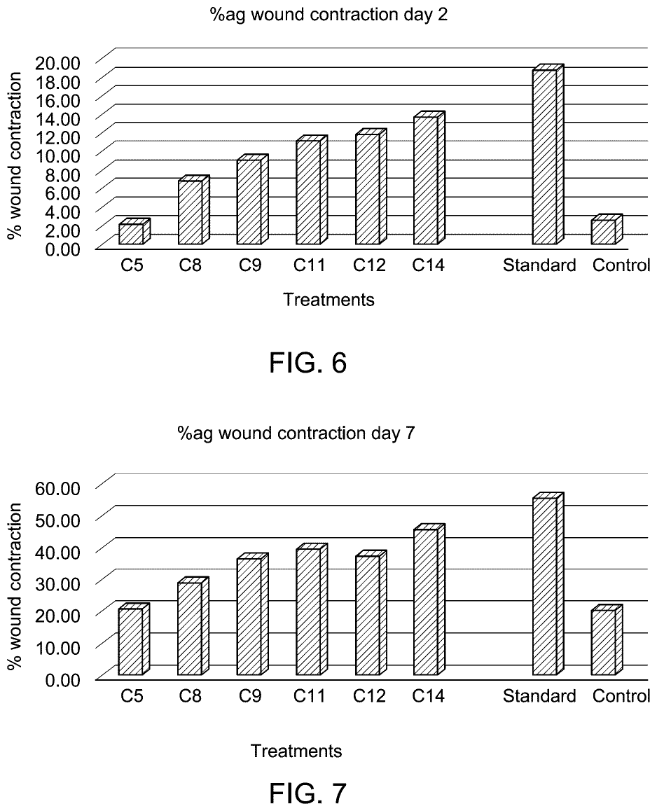

FIG. 6 shows the mean±standard deviation (sd) of the percent wound contraction on the second day. Observation of wounds on second day showed the percent wound healing in the control sample was less than average with a contraction of 2.6%, as shown in FIG. 6. The percent wound contraction (% WC) of the standard sample was highest with an average contraction of 19%. Wound contraction ability of the other treatments was found to be less than the standard sample, whereas C5 (formula 31) showed less wound contraction ability than the control sample.

On the seventh day of treatment, the wounds had contracted more, a 20% wound contraction in the animal which received no treatment. A 55% wound contraction was observed in the standard sample. FIG. 7 depicts the mean±sd of percent wound contraction at 7th day. C14 (formula 36) showed a 45% wound contraction which was similar to the standard sample (53%), as shown in FIG. 7.

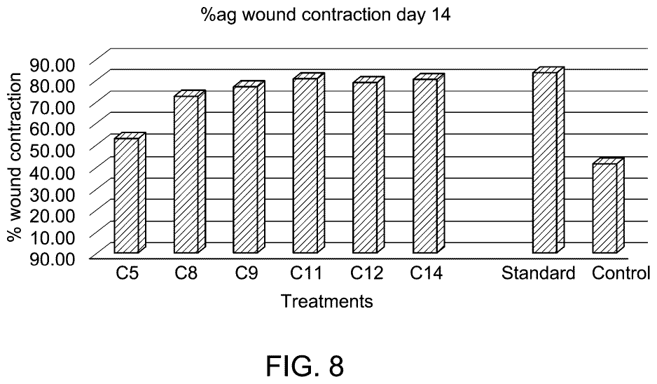

FIG. 8 shows mean±sd of percent wound contraction at 14th day. Benzimidazolium-based mononuclear Ag(I) complex C5 (formula 31) shows an average wound contraction of 53%, which is better than the control sample with a would contraction of 41%. Imidazolium-based dinuclear Ag(I) complex (C8) displays an average wound contraction of 73%. All benzimidazolium-based dinuclear Ag(I) complexes show an average wound contraction percentage in the range of 77-80%, as shown in FIG. 8. The wound contraction ability of hexanuclear Ag(I) complex may be close to that of the standard sample (83%) on the basis of results of a group of tetranuclear Ag(I) complexes [Riaz, A. et al., Synthesis of sandwich type acyclic tetra-nuclear silver(I)-N-heterocyclic carbene complexes for wound healing applications, Z Naturforsch C J Biosci, 2020, 75, 9-10, 369-376, which is incorporated herein by reference in its entirety]. A lesser wound contraction percentage of synthesized silver complexes of the present disclosure as compared to that of the standard sample may be due to standard formulation under varying conditions.

### Example 22: Fourier-Transform Infrared Spectroscopy (FTIR) of Azolium Salts and Relevant Ag-NHC Complexes

For salts (S1-S19) strong and sharp peaks in the range of 3400-3415 wavenumbers (cm⁻¹) appeared not due to stretching vibrations of N on azolium ring (Cₐₗᵢₚₕ-N), but due to hygroscopic nature of the salts, as most of the peaks were overlapped by water peaks. Bis-imidazolium salts like S8 (formula 22) showed peaks at 3442 cm⁻¹ because of non-overlapping H₂O. Multinuclear-benzimidazolium salts did not show peaks in the region of 3400-3450 cm⁻¹, depicting the absence of overlapping water molecules. Peaks in the range of 28003000 cm⁻¹ were observed due to C—H stretching vibrational bands (Cₐₗᵢₚₕ-H). Pure modes of C—N stretching vibrations appeared in the range of 1300-1500 cm⁻¹. Vibrational bands around 1020-1220 cm⁻¹ were labelled as other ring vibrations. The “four fingers (ffs)” pattern in the region of 1350-1500 cm⁻¹ was due to silver complex formation.

### Example 23: Nuclear Magnetic Resonance (NMR) Spectroscopy of Azolium Salts and its Ag(I) Complexes

Proton nuclear magnetic resonance (1H NMR) of mono-imidazolium salts S1-S2 (formula 11) showed a singlet around 8.40 to 4.50 parts per million (ppm) which was assigned as acidic proton peak of NCHN while the same peaks for S6 was observed at 8.75 ppm.

For mono- and di-azolium salts, a peak for an acidic proton appeared in the range of 10.11-11.74 ppm. Salt S14 (formula 25) with PF₆ counterpart was observed at 9.66 ppm. Acidic protons of multinuclear-benzimidazolium salts S17 and above appeared in the range of 10.00-10.28 ppm as more than one singlet peak. When multinuclear-NHC salts were converted into the relevant Ag-NHC complexes, a change in chemical shift values was seen. The signal for methyl protons, shifted to more up-field positions in the range of −0.42 to −0.25 ppm.

A set of resonance bands in the range of 6.81-9.00 ppm was assigned to protons in an aromatic region. The spectral range of 5.10-5.91 ppm indicated the presence of benzylic protons. In case of multinuclear-benzimidazolium salts, resonance signals for 8 ethylenic protons were observed in the range of 5.20-5.23 ppm, with those for benzylic protons appearing in the region of 5.69-5.79 ppm. Aliphatic protons gave characteristic up-field peaks in the range of 1.10-4.60 ppm. Terminal protons of alkyl chains were observed near 0.81-1.05 ppm. Resonance peaks showed negligible shifting in their chemical shift values.

Carbon-13 nuclear magnetic resonance (¹³C NMR) spectra of the salts showed chemical shifts around 140.6-143.8 ppm, which is characteristic for salts. In the case of mono-imdazolium salts, carbenic peaks were observed in the range of 135.9-136.7 ppm. Other spectral ranges were assigned as follows: 121-129 ppm for imidazolium carbons S1-S3 (formula 11), 121-133 ppm for bis-imidazolium carbons, 124-138 ppm for aromatic carbons, 46-67 ppm for benzylic carbons, 19-35 ppm for aliphatic carbons, and 10-14 ppm for terminal alkyl carbons. ¹³C NMR of Ag complexes appeared with a characteristic peak in the range of 178-190 ppm, which was assigned as the carbene carbon. Due to the binding of the carbene carbon with two silver isotopes, ¹⁰⁷Ag and ¹⁰⁹Ag, the carbene carbons gave a peak of doublets (per NHC-Ag) in the structure of dimeric complexes.

### Example 24: Crystal Structure Analysis of Complexes

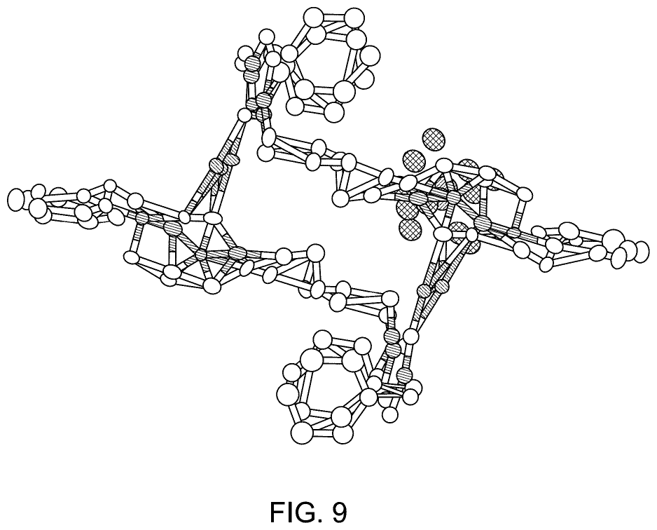

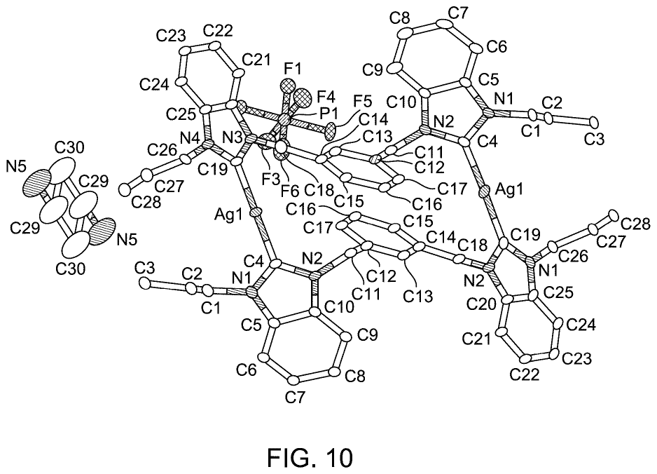

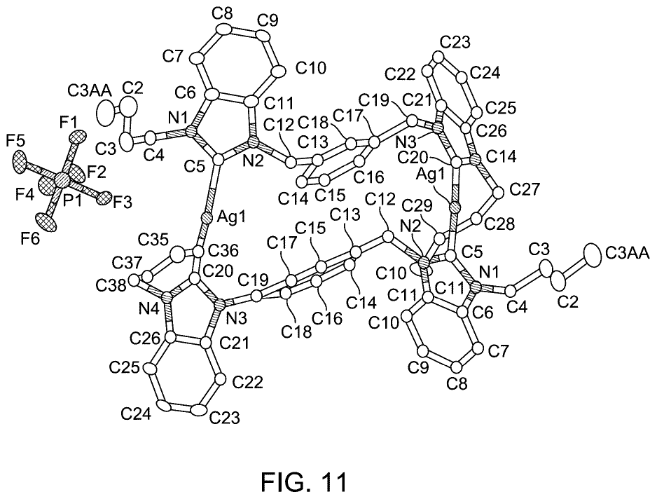

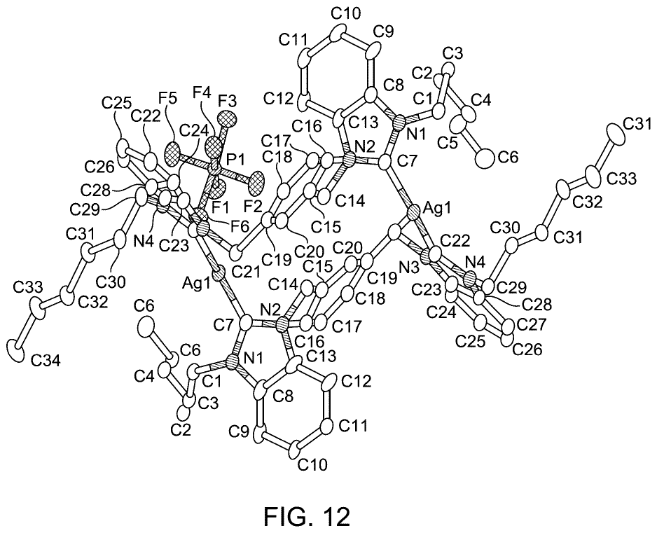

Single crystals of Ag(I)-NHC were grown by either a slow evaporation or a slow diffusion method of crystallization. Four bis-benzimidazolium-Ag(I) complexes were characterized by X-ray diffraction (XRD) analysis. FIG. 9 shows a crystal structure of complex 8. Bis-imidazolium based Ag-NHC complex C8 was crystallized in triclinic space group P1, containing 2 PF₆ counter anions, as shown in FIG. 9. FIGS. 10-12 depict a crystal structure of complex 9, complex 10, and complex 11, respectively. Crystal data details of complexes C8-C₁₁ are given in Table 1.

TABLE 1
Crystal data details of bis-azolium-Ag(I) complexes (C8-11)

| | C8 | C9 | C10 | C11 |
|---|---|---|---|---|
| Formula | C₂₅H₂₈AgF₆N₄P | C₅₆H₆₀Ag₂F₁₂N₈P₂ | C₆₀H₆₈Ag₂F₁₂N₈P₂ | C₆₈H₈₄Ag₂F₁₂N₈P₂ |
| Formula weight | 637.36 | 1350.82 | 1406.92 | 759.57 |
| (g/mol) | | | | |
| Crystal system | triclinic | monoclinic | monoclinic | triclinic |
| Space group | P1 | P2₁/n | C2/c | P1 |
| Unit cell | | | | |
| dimensions | | | | |
| a (Å) | 9.5869 (3) | 11.4913 (4) | 19.1701 (6) | 9.3846 (4) |
| b (Å) | 12.3049 (4) | 16.3548 (6) | 16.7047 (5) | 11.4566 (4) |
| c (Å) | 12.4159 (4) | 15.6404 (6) | 20.2230 (6) | 16.0758 (6) |
| α (°) | 98.5570 (10) | 90 | 90 | 77.1820 (10) |
| β (°) | 110.6530 (10) | 104.8270 (10) | 106.3220 (10) | 85.943 (2) |
| γ (°) | 97.6890 (10) | 90 | 90 | 86.2820 (10) |
| V (cubic | 1327.52 (8) | 2841.55 (18) | 6215.0 (3) | 1678.96 (11) |
| angstroms (Å³)) | | | | |
| Z | 2 | 2 | 4 | 2 |

### Example 25: Crystal Structure Analysis of Complexes

Sodium carbonate (Na₂CO₃) and KOH were purchased from Merck (Germany). All the alkyl halides and benzimidazole were purchased from Sigma (England). Potassium hexaflorophosphate was purchased from Fluka (England). All additional chemicals and reagents were bought from Sigma Aldrich and utilized without further purification. FTIR spectral analysis was performed using a Perkin-Elmer 2000 spectrophotometer. The Bruker Avance 300 MHz machine operating at 296 K was used for NMR spectroscopy.

### Example 26: Antioxidant Test

Salts (S1-S19) and their silver complexes (C1-C₁₆) as potential antioxidants were determined by an in vitro DPPH assay. Three different compound solutions with concentrations of 200 μM, 100 μM, and 50 μM in DMSO, and ascorbic acid in distilled water as a positive control, were prepared. 100 microliters (μL) of a DPPH solution and 100 μL of each sample was pipetted into each well of a 96-well plate, with four wells per one sample. Two negative controls, 100 μL DPPH+100 μL DMSO (negative control 1) and 100 μL DPPH+100 μL water (negative control 2), were added to wells of the 96-well plate with four wells per sample. Plates were incubated for 20-30 minutes (min) at 37 degrees Celsius (° C.), and absorbance of the DPPH tincture was measured at 517 nanometers (nm) on a microplate enzyme-linked immunosorbent assay (ELISA) reader.

The present disclosure describes multinuclear silver complexes exhibiting wound contraction abilities. Additionally, it highlights that benzimidazolium-based bis-NHC silver complexes demonstrate greater wound contraction abilities compared to their imidazolium-based bis-NHC counterparts. Furthermore, the present disclosure asserts that these bis-NHC silver complexes show activity comparable to that of commercially available wound contracting gels, indicating their use as effective wound healing agents.

According to the present disclosure, sterically tuned tetra- and hexa-nuclear metal NHC complexes are described and used in wound healing. Ag—NHC may be made from two different NHC precursors: imidazolium and benzimidazolium ligands. Four precursors (P₁, P₂ₐ-P_{2c}) were synthesized and used as linkers to form multinuclear-NHC salts. Methods of synthesizing NHC-based silver complexes using silver oxide as a base is described. A method for treating cancer cells using an effective amount of silver-NHC complexes and inhibiting solid tumor growth by inhibiting vessel growth is also described. It is observed that benzimidazolium-based bis-NHC silver complexes showed better wound contraction than imidazolium-based bis-NHC silver complexes. Some bis-NHC silver complexes displayed activity comparable to a commercially available wound contracting gel, with, multinuclear silver complexes showing good wound contraction ability compared to mononuclear complexes. The present disclosure offers a method to treat wounds by administration of Ag-NHC complexes.

Numerous modifications and variations of the present disclosure are possible in light of the above teachings. It is therefore to be understood that within the scope of the appended claims, the disclosure may be practiced otherwise than as specifically described herein.

1: A complex, including:
at least two N-heterocyclic carbene (NHC) ligands with the formula I: 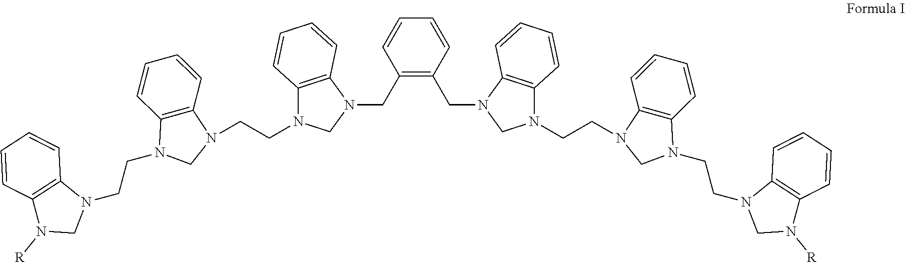
wherein R is selected from a group consisting of a propyl functional group, a butyl functional group, and a benzyl functional group, and
two to six silver atoms,
wherein each of the silver atoms are bonded to the at least two NHC ligands.

2: The complex of claim 1, wherein the NHC ligand of formula I is made by a process, including:
refluxing a 1-X-1H-benzimidazole with a 1,2-dibromoethane to form a 3-(2-bromoethyl)-1-X-1H-benzoimidazolium,
wherein X is selected from a group consisting of propyl, butyl, and benzyl,
refluxing a 1,3-bis((1H-imidazol-1-yl)methyl)benzene with the 3-(2-bromoethyl)-1-X-1H-benzoimidazolium in an organic solution to form a tetrakis-benzamidazolium precursor;
refluxing the tetrakis-benzamidazolium precursor with 3-(2-bromoethyl)-1-X-1H-benzoimidazolium in 1,4-dioxane to form a salt of formula 1.

3: The complex of claim 2, wherein X is propyl and the tetrakis-benzamidazolium precursor has a percent yield of 45 to 50% based on an initial weight of reactants.

4: The complex of claim 2, wherein X is butyl and the tetrakis-benzamidazolium precursor has a percent yield of 42 to 48% based on an initial weight of reactants.

5: The complex of claim 2, wherein X is benzyl and the tetrakis-benzamidazolium precursor has a percent yield of 45 to 50% based on an initial weight of reactants.

6: The complex of claim 1, wherein the complex is made by a process including:
mixing the NHC ligand of formula I with a silver salt in a polar solvent in the absence of light for 45 to 50 hours (h) to form a mixture;
filtering the mixture;
collecting a filtrate; and
mixing a hexafluorophosphate salt with the filtrate in a polar solvent to form the complex.

7: A method of healing wounds, including:
administering the complex of claim 1 to an open wound; and
closing the open wound to form a healed wound,
wherein a surface area of the healed wound is smaller compared to an initial surface area of the open wound.

8: The method of claim 7, wherein the administering the complex to the open wound occurs at least 2 times a day.

9: The method of claim 7, wherein the administering the complex to the open wound occurs for at least 1 to 15 days.

10: The method of claim 7, wherein after the administering the complex to the open wound and the closing the open wound, the wound heals with a percent wound contraction of 70 to 85% based on the initial surface area of the open wound.

11: A method of anti-angiogenesis, including:
administering the complex of claim 1 to a subject; and
reducing growth of blood vessels in the subject.

12: The method of claim 11, further including:
dissolving blood vessels during the reducing.

13: The complex of claim 1, further including:
at least two NHC ligands with the formula II:

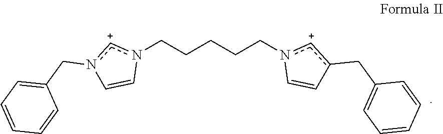

14: The complex of claim 1, further including:
at least two NHC ligands with the formula III: 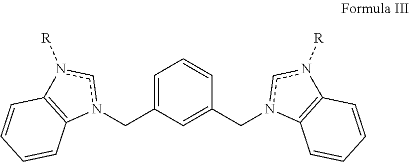
wherein R is selected from a group consisting of a propyl functional group, a butyl functional group, a hexyl functional group, a benzyl functional group, and an ethyl phenyl functional group.

15: The complex of claim 1, further including:
at least two NHC ligands with the formula IV: 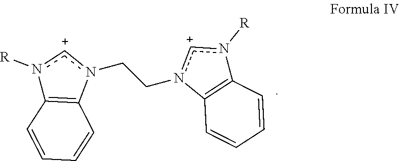
wherein R is a diphenyl methane functional group.

16: The complex of claim 13, wherein the complex has a percent yield of 75 to 85% based on an initial weight of reactants.

17: The complex of claim 14, wherein the complex has a percent yield of 65 to 90% based on an initial weight of reactants.

18: The complex of claim 15, wherein the complex has a percent yield of 80 to 85% based on an initial weight of reactants.

19: The complex of claim 14, wherein the functional group is the hexyl functional group, and the complex has a unit cell volume of 1650 to 1700 cubic angstroms (Å³).

20: A pharmaceutical composition, including:
the complex of claim 1;
a filler;
a stabilizer;
a surfactant;
a binder;
a glidant; and
a lubricant.
